(19) United States
(12) Patent Application Publication
(10) Pub. No.: US 2007/0100245 A1
(43) Pub. Date: May 3, 2007

(54) LASER BLOOD FLOW IMAGING APPARATUS

(75) Inventor: Susumu Kashima, Fuchu-Shi (JP)

Correspondence Address: BURR & BROWN, PO BOX 7068, SYRACUSE, NY, 13261-7068, US

(73) Assignee: Omegawave, Inc., Fuchu-Shi (JP)

(21) Appl. No.: 11/553,619

(22) Filed: Oct. 27, 2006

(30) Foreign Application Priority Data
Nov. 2, 2005 (JP) 2005-319297

(51) Int. Cl. A61B 5/02

(52) U.S. Cl. 600/504

(57) ABSTRACT

Provided is a laser blood flow imaging apparatus whose configuration is simple and which has functions of arithmetically and concurrently processing blood flows of all points of measurement. The laser blood flow imaging apparatus includes: a laser light irradiating unit for irradiating laser light of a predetermined wavelength to a blood flow within a vital tissue; an image capturing unit for capturing scattered light from the blood flow irradiated with the laser light; an image processing unit for processing an image of the captured blood flow per pixel; a value-of-blood flow calculating unit for calculating a value of blood flow based on data per the pixel and for processing as a color discernible image corresponding to the calculated value; and a display unit for displaying the image of the blood flows processed by the value-of-blood flow calculating unit as a two-dimensional color image.

### BACKGROUND OF THE INVENTION

1. Field of the Invention

The present invention relates to a technology of medical instrumentation, and more specifically, to a technology for inspecting a state of vital tissues by means of a blood flow meter utilizing laser light.

2. Description of the Related Art

Oxygen and nutrition are supplied to vital tissues through blood flows. Specifically, they are supplied to the vital tissues substantially through microcirculation, and it is important to obtain information on blood flows thereof. There has been known a blood flow meter utilizing laser light as an instrument for measuring a state of blood flows.

The blood flow meter (laser blood flow meter) utilizing laser light makes use of such characteristics of the laser light that frequency of the laser light scattered by red blood cells flowing within blood vessels is modulated while frequency of the laser light scattered by static tissues of the vital tissues is not modulated. There are fiber-optic-type and scan-type instruments as a conventional instrument for measuring blood flows of vital tissues by utilizing the laser light.

For example, as shown in FIG. 2, a fiber-optic-type laser blood flow meter 1 has a blood flow meter probe 2 having an irradiation fiber 2a for irradiating the laser light to a vital tissue T from a laser light source 1a and a light receiving fiber 2b for receiving part of the laser light irradiated from the irradiation fiber 2a and scattered within the vital tissue.

After carrying out photo-electric conversion of the laser light scattered within the vital tissue, an arithmetic processing section 1b provided to a main body side of the laser blood flow meter finds a volume of blood flow of the vital tissue by arithmetic operation based on the characteristics that flow speed of the blood cell is proportional to the modulated frequency and that the volume of the blood cells depends on a quantity of frequency modulated light.

At this time, the irradiation of the laser light and reception of the transmitted light or the scattered light may be carried out by providing a photo-detector such as a semiconductor laser and a photo-diode at a tip of the probe, as well as by using the fiber-optic probe.

Incidentally, the fiber-optic-type meter irradiates the laser light to the vital tissue through the optical fiber and receives the scattered light from the vital tissue by means of paired optical fibers to guide the light to the photo-detector. After receiving the light by the photo-detector, the meter arithmetically processes to calculate a value of blood flow. This fiber-optic-type meter can measure basically only the blood flow of one point immediately under the optical fiber and needs to compose a set of many optical fibers and to measure blood flow of each point before forming an image in order to obtain an image of blood flow by the multi-point measurement. However, it is possible to obtain values of blood flow of respective points concurrently in real-time in this case.

Meanwhile, the scan-type meter irradiates laser light directly to the vital tissue from a distant place and receives scattered light from the tissue by a photo-detector installed at a distant place. When intensity of the received light is weak at this time, the meter is provided with a lens in front of the photo-detector to assure enough intensity of the received light. The meter arithmetically processes the scattered light received by the photo-detector by an arithmetic circuit to calculate (operate) a value of blood flow. This operation method is basically the same with the fiber optic-type meter and the meter carries out the arithmetic processing by receiving the scattered light while scanning the laser light on the vital tissue sequentially point by point to calculate the value of blood :flow. After scanning, the meter displays a two-dimensional image of the blood flows as changes of color based on the value of blood flow of each point. Because a measurement time of the scan-type meter is normally several minutes, the meter has a problem in that time lag occurs in calculating the value of blood flow of each point and that the vital tissue to be measured itself moves during scanning.

[Non-patent Document] “Studies of Detached Observation Method of Blood Flow of Capillary and Blood Flow of Vascular Floor of Arteriola and Venula by Laser Vital Tissue Blood Flow Meter” written by Susumu Kashima et. al., Japanese Society of Medical Instrumentation 66, 307-313, 1996

As described above, the fiber-optic-type meter that aims to obtain an image of blood flows by carrying out the multi-point measurement requires optical fibers whose number corresponds to a size of an object to be measured. The fiber-optic-type meter also requires arithmetic processors for calculating the blood flow of the respective points as many as the number of points of measurement, which leads to a problem in that the size of the meter itself increases.

Meanwhile, the scan-type blood flow meter requires much time to scan the object to be measured by the laser light, which may result in an incorrect relationship between value of blood flows of the first and last points of measurement due to a large interval of the measuring time. There also arises another problem in that if the object to be measured moves during measurement, reliability of the point of measurement itself is lost.

### SUMMARY OF THE INVENTION

The present invention has been made to solve the aforementioned technological problems, and a technological object of the invention is to provide a laser blood flow imaging apparatus whose configuration is simple and which has functions of arithmetically and concurrently processing blood flows of all points of measurement.

In order to achieve the above-mentioned object, the present invention adopts the following means.

That is, a laser blood flow imaging apparatus according to an aspect of the present invention includes:

laser light irradiating means for irradiating laser light of a predetermined wavelength to a blood flow within a vital tissue;

image capturing means for capturing scattered light from the blood flow irradiated with the laser light;

image processing means for processing an image of the captured blood flow per pixel;

value-of-blood flow calculating means for calculating a value of blood flow based on data per the pixel and :for processing as a color discernible image corresponding to the calculated value; and

display means for displaying the image of the blood flows processed by the value-of-blood flow calculating means as a two-dimensional color image.

Further, in a laser blood flow imaging apparatus according to another aspect of the invention, the image capturing means has an optical filter for transmitting light of one of the wavelength of the laser light and a narrow wavelength width centering on the wavelength. This configuration allows light coming from the outside, other than that of the measuring wavelength, to be blocked.

Further, in a laser blood flow imaging apparatus according to another aspect of the invention, the image capturing means has a polarizer for capturing only component vertical to a polarizing direction of the laser light to remove surface reflection light from the vital tissue irradiated with the laser light and to receive only the light scattered within the vital tissue.

Further, in a laser blood flow imaging apparatus according to another aspect of the invention, the laser light irradiating means has irradiation area adjusting means for adjusting an irradiation area of the laser light corresponding to a measuring range of the vital tissue.

Further, in a laser blood flow imaging apparatus according to another aspect of the invention, the image capturing means has image capturing area adjusting means for adjusting an image capturing area corresponding to the measuring range of the vital tissue.

Still more, a laser blood flow imaging apparatus according to another aspect of the invention further includes visible light irradiating means for irradiating visible light that allows an irradiation region of the laser light to be discriminated concurrently with the laser light. This configuration allows the irradiation region of the measuring laser light to be discriminated when the measuring laser light has a wavelength of invisible light such as a near infrared region.

As described above, the invention has the following effects.

(1) It is possible to vary the irradiation and an image capturing area corresponding to the measuring area of the vital tissue to be measured, thereby eliminating waste and allowing display of a high-resolution color image of blood flows when the measuring area is small.

(2) It is possible to visually confirm the irradiation surface by irradiating the visible light concurrently with the laser light to the irradiation surface even when the measuring laser light is invisible.

(3) It is possible to efficiently receive only the measuring light and to improve a signal-to-noise ratio by providing the optical filter in front of the image capturing means such as the CCD camera for capturing images.

(4) It is possible to carry out the highly sensitive measurement of blood flows by receiving no surface reflection light from the vital tissue and by receiving only the light scattered within the vital tissue by providing the polarizer in front of the image capturing means such as the CCD camera for capturing images.

(5) The reliability in comparing values of blood flow of respective points is improved because positional relationship of the respective points is unchangeable and there is no time lag in the measurement of blood flows by irradiating the laser light to the entire surface of the measuring face, capturing images at a time, and then carrying out the arithmetic operation on the blood flows.

Accordingly, according to the invention, it is possible to provide the laser blood flow imaging apparatus whose configuration is simple, which can vary the irradiation area of the laser light and the light receiving or image capturing area corresponding to the size of the vital tissue to be measured, and which has the function of arithmetically and concurrently processing blood flows of all measuring points.

### BRIEF DESCRIPTION OF THE DRAWINGS

In the accompanying drawings:

FIG. 1 is a perspective view of an outline of a laser blood flow imaging apparatus of the invention;

FIG. 2 is a conceptual block diagram of the laser blood flow imaging apparatus of the invention; and

FIG. 3 shows an example of displayed images of a display unit.

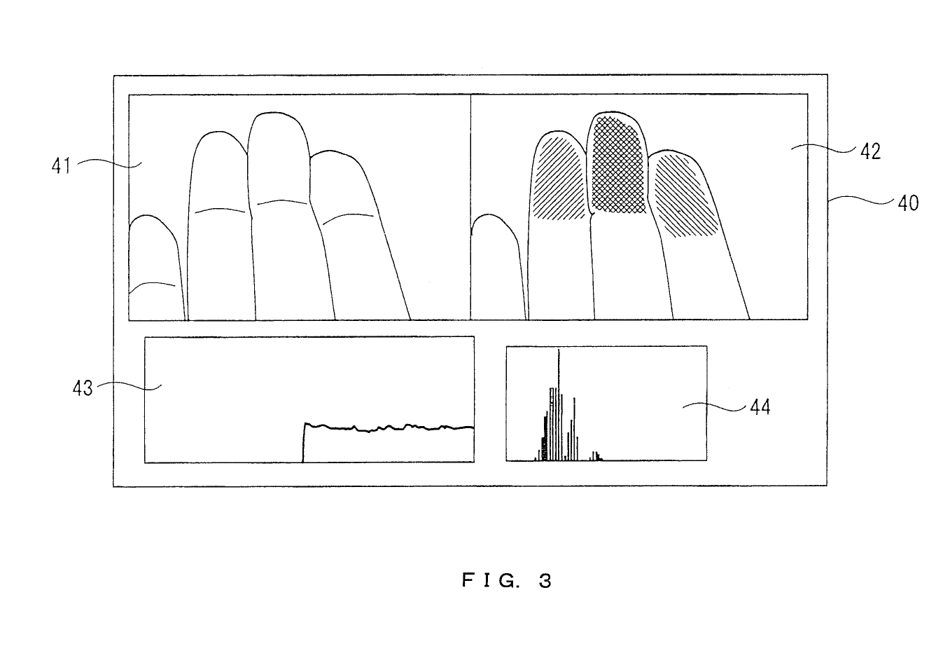

### DETAILED DESCRIPTION OF THE PREFERRED EMBODIMENT

A best mode for carrying out a laser blood flow imaging apparatus of the invention will be illustratively explained in detail with reference to the drawings. The laser blood flow imaging apparatus will be explained as a two-dimensional laser blood flow meter in this embodiment.

[Outline of Laser Blood Flow Imaging Apparatus]

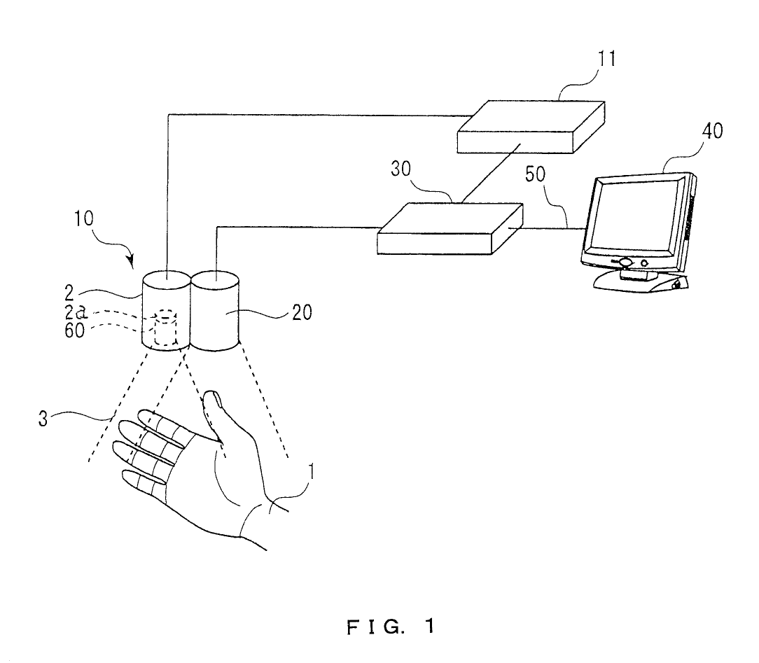

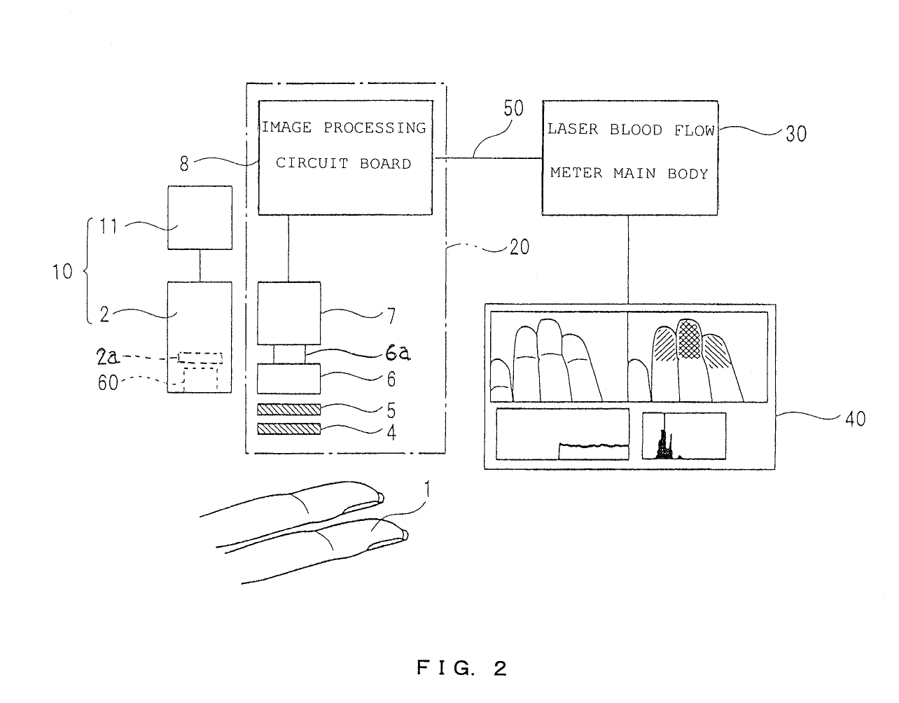

That is, as shown in FIGS. 1 and 2, the two-dimensional laser blood flow meter of this embodiment has laser light irradiating means 10 for irradiating laser light 3 of a predetermined wavelength to blood flows within a vital tissue 1, image capturing means 20 for capturing scattered light of the blood flow to which the laser light 3 has been irradiated, image processing means 8 for processing a captured image of the blood flows per pixel, value-of-blood flow calculating means (i.e., a laser blood flow meter main body) 30 for calculating the value of blood flow based on data per pixel and for processing as a color discernible image corresponding to the calculated values, and display means 40 for displaying the image of the blood flows processed by the value-of-blood flow calculating means 30 per color as a two-dimensional color image. It should be noted that the laser light irradiating means 10, the image capturing means 20, the image processing means 8, the value-of-blood flow calculating means 30, and the display means 40 are wired by a signal line 50 capable of transmitting/receiving image signals.

[Laser Light Irradiating Means 10]

The laser light irradiating means (i.e., laser unit) 10 has a semiconductor laser (i.e., laser light source) 2 for irradiating the laser light 3, and a laser light driving unit 11. The laser light driving unit 11 drives and controls the laser light source 2 to irradiate the laser light 3 to the whole range of the vital tissue 1 to be measured at once. It should be noted that the laser blood flow meter proper 30 composed of a computer as described later arithmetically processes the light scattered by the tissue as a value of blood flow. The laser light irradiating means 10 is also provided with irradiation area adjusting means 2a for directly irradiating the laser light 3 from the semiconductor laser (i.e., laser light source) 2 or the like to the vital tissue 1, or for irradiating the laser light 3 to the whole area of the measuring range by expanding the irradiation range of the laser light 3 by a lens or the like corresponding to the dimension of the measuring range of the vital tissue 1. The irradiation area adjusting means 2a is a lens or the like for adjusting the irradiation area to correspond to the size of the vital tissue 1, and is built in at the tip of the laser light source 2.

Incidentally, there is a case where the measuring laser light 3 has a wavelength of invisible light such as a near infrared region. That is, the irradiation surface is invisible by naked eyes if the laser light 3 is the near infrared light that is unlikely to be influenced by a difference of oxygenating and de-oxygenating states of red blood cells.

[Visible Light Irradiating Means 60]

The two-dimensional laser blood flow meter of this embodiment is provided with a visible light irradiating unit (i.e., visible light irradiating means) 60 for irradiating visible light, which allows the irradiation region of the measuring laser light 3 to be discriminated, to the center of the irradiation range, to an area in the very vicinity thereof, or to the whole irradiation surface of the measuring laser light 3 concurrently with the measuring laser light 3.

[Image Capturing Means 20]

The image capturing means 20 captures the scattered light from the vital tissue 1 to which the laser light 3 has been irradiated by an image capturing device 7 such as a CCD camera via a lens 6. The image capturing means 20 has, in front of the lens 6 or between the lens 6 and the image capturing device 7, a polarizer 5 for capturing only a component vertical to a polarizing direction of the irradiated light, and an optical filter 4 for selectively transmitting the measuring laser light 3 of the wavelength.

[Polarizer 5]

The polarizer 5 removes influence of surface reflection light from the vital tissue 1, and then the image capturing device 7 removes the surface reflection light as noise from the vital tissue 1 to which the laser light 3 has been irradiated through the polarizer 5, and captures the scattered light from the vital tissue 1 of only the component vertical to the polarizing direction of the irradiated light.

[Optical Filter 4]

The optical filter 4 selectively transmits light of the wavelength of the measuring laser light 3 or that of narrow wavelength width centering on that wavelength. That is, the image capturing device 7 of this embodiment can remove the surface reflection light from the vital tissue 1 by using the polarizer 5, and can block light having a wavelength other than the measuring wavelength coming in from the outside and the visible light for confirming the irradiation surface by using the optical filter 4.

[Image Capturing Area Adjusting Means 6a]

The image capturing means 20 also has image capturing area adjusting means 6a for freely adjusting an image capturing area corresponding to a measuring range of the vital tissue 1. The image capturing area adjusting means 6a may be a device that allows the lens 6 to be replaced depending on the size of the vital tissue 1 that is the object of the measurement, or may use a magnification converting lens such as a zoom lens.

[Image Processing Means]

Image processing means has the image processing circuit board 8 for processing the image of the blood flows captured by the image capturing device 7 per pixel. The image processing circuit board 8 converts the image-processed image signal into image data that can be processed by the laser blood flow meter proper 30 to transmit to the laser blood flow meter proper 30. It should be noted that the image processing means may be built in or built separately from the image capturing means 20, e.g., the CCD camera.

[Value-of-Blood Flow Calculating Means 30]

The laser blood flow meter proper 30 that is the value-of-blood flow calculating means has software for calculating the value of blood flow recorded in the apparatus composed of the computer and the laser blood flow meter proper 30, and software for converting the data into the color discernible image. The laser blood flow meter proper 30 calculates the value of blood flow based on the data per pixel according to procedures of the software for calculating value of blood flows, processes the calculated value as the color discernible image in accordance to procedures for converting the calculated values into the color discernible image, and converts the processed color discernible image into data that can be displayed on the display means (i.e., display unit 40). For example, the laser blood flow meter proper 30 carries out signal processing such as temporal change of intensity of each pixel of the image capturing device 7.

[Principle of Calculation of Value of Blood Flow]

Next, a dynamic speckle analytical method will be explained as an example of the principle of calculation of the value of blood flow that is the procedure of the software for calculating the value of blood flow.

A grain like pattern called a speckle pattern occurs when light (i.e., coherent light) whose phases are equal, such as the laser light 3, is irradiated to a rough surface. This speckle pattern results from superposition of laser scattered light from respective places and occurs because the lights whose phases are different overlap one another. For example, the speckle pattern does not change and the intensity of light does not fluctuate when the laser light is irradiated to a stationary object.

However, the speckle pattern fluctuates temporally in case of a moving object. Speed of this fluctuation depends on speed of the moving object. Accordingly, it is possible to find the speed of the moving object by checking a degree of temporal changes of the intensity (i.e., intensity of light) of the speckle.

Because the light reaches to the inside of the vital tissue 1 when the laser light 3 is irradiated to the vital tissue 1, the speckle pattern changes in accordance with the movement of red blood cells.

The device to which this principle is applied is a speckle blood flow meter.

Here, a value of blood flow BF, for example, may be found as follows.
BF=SAc(1/1sd(x,y))c
where, in the equation, “Ac” denotes a constant corresponding to a multiplier member C, “1sd(x, Y)” denotes fluctuation of the intensity of captured received light of the pixel (x, y), and “C” denotes a multiplier.
[Display Means]

The display means has a display unit 40 for displaying blood flows as a color image indicating the blood flows per color corresponding to the values thereof. The display unit 40 is divided into four region: a region 41 for displaying a normal image in color; a region 42 for displaying blood flows in the tissue in color; a region 43 for displaying time of average blood flow in a line graph; and a region 44 for displaying a histogram. The display unit 40 displays the respective images in the respective regions based on control of the value-of-blood flow calculating means 30.

Next, operations of the two-dimensional laser blood flow meter of this embodiment will be explained.

The two-dimensional laser blood flow meter captures part of the laser light 3 scattered by the vital tissue 1 by the image capturing device 7 after irradiating the laser light.

At this time, the optical filter 4 functions to selectively transmit light of the wavelength of the measuring laser light 3 or of a narrow wavelength width centering on that wavelength so as not to capture the light from the outside other than the measuring wavelength and the visible light for confirming the irradiation surface. The polarizer 5 functions to avoid capturing of the laser light 3 reflected by the surface of the vital tissue 1. It should be noted that the surface reflection light does not contain information on the blood flow because it is reflected by the very surface of the vital tissue 1. Therefore, the image capturing device 7 captures the scattered light from the vital tissue 1 of only the component vertical to the polarizing direction of the laser light 3 by the polarizer 5 by utilizing characteristics that the surface reflection light has the same polarizing direction with the laser light 3 and that the scattered light scattered within the vital tissue 1 becomes the component vertical to the polarizing direction of the laser light 3.

The lens 6 may be replaced so as to effectively capture only the measuring range or may be arranged so as to be able to select magnification by using the magnification changing lens such as the zoom lens.

Signal light captured by the image capturing device 7 is sent to the image processing circuit board 8 via the signal line 50 per pixel. Then, the signal per pixel processed by the image processing circuit board 8 is sent to the laser blood flow meter proper 30. The laser blood flow meter proper 30 analyzes the signal by the software (i.e., value-of-blood flow calculating means) for calculating blood flows and converts the value of blood flows into colors corresponding to the values into the two-dimensional laser blood flow meter that can be displayed by the display unit 40. At this time, blue to red colors, for example, may be used as colors corresponding to the value of blood flow from to low to high values.

According to this embodiment, it is possible to display the two-dimensional blood flow image in color by irradiating the laser light 3 to the vital tissue 1, changing the speckle state corresponding to the flow of red blood cells, capturing this state by the image capturing device (CCD camera) 7, and analyzing and calculating it by the laser blood flow meter proper 30.

Note that it is possible to provide a two-dimensional image laser blood flow meter (i.e., laser blood flow imaging apparatus) that realizes speed of one image in about one second by using consecutive images and high-speed analyzing process of a highly sensitive CCD camera. Further, this embodiment has the following operations and effects.

(1) Highly sensitive measurement: Speed for measuring an image of blood flow of one time is within one second and changes of distribution of blood flows may be observed almost in real-time.

(2) High resolution: Because the CCD camera calculates the blood flows per pixel, it is possible to display the blood flows extremely finely.

(3) Data analysis: It is possible to calculate the average volume of blood flows by arbitrarily specifying a partial region and to display temporal changes in waveform in the region 43. Still more, it is possible to analyze the distribution of blood by displaying the histogram in the region 44.

(4) Non-contact measurement: It is possible to measure without touching a living body, thereby giving no stress and achieving excellence in its reproducibility.

(5) Display in color. Because it displays blood flows of tissue of each region in color as the digital image, distribution of blood flows may be clearly measured.

(6) Reliability: Because each pixel of the image of blood is information of identical time, no time lag occurs differing from scanning per point.

(7) Convenience: It is useful in deciding the measuring region to display regions concurrently by a video function of the CCD camera. Still more, it is possible to readily make an anatomical judgment because the blood flow is synchronized with the color image of the blood flow in measurement thereof.

(8) Small size: It is possible to downsize the apparatus because the measuring section is only the CCD camera and the laser unit.

1. A laser blood flow imaging apparatus, comprising:
laser light irradiating means for irradiating laser light of a predetermined wavelength to a blood flow within a vital tissue;
image capturing means for capturing scattered light from the blood flow irradiated with the laser light;
image processing means for processing an image of the captured blood flow per pixel;
value-of-blood flow calculating means for calculating a value of blood flow based on data per the pixel and for processing as a color discernible image corresponding to the calculated value; and
display means for displaying the image of the blood flows processed by the value-of-blood flow calculating means as a two-dimensional color image.

2. A laser blood flow imaging apparatus according to claim 1, wherein the image capturing means has an optical filter for transmitting light of one of the wavelength of the laser light and a narrow wavelength width centering on the wavelength.

3. A laser blood flow imaging apparatus according to claim 1, wherein the image capturing means has a polarizer for capturing only component vertical to a polarizing direction of the laser light to remove surface reflection light from the vital tissue irradiated with the laser light and to receive only the light scattered within the vital tissue.

4. A laser blood flow imaging apparatus according to claim 1, wherein the laser light irradiating means has irradiation area adjusting means for adjusting an irradiation area of the laser light corresponding to a measuring range of the vital tissue.

5. A laser blood flow imaging apparatus according to claim 1, wherein the image capturing means has image capturing area adjusting means for adjusting an image capturing area corresponding to the measuring range of the vital tissue.

6. A laser blood flow imaging apparatus according to claim 1, further comprising visible light irradiating means for irradiating visible light that allows an irradiation region of the laser light to be discriminated concurrently with the laser light.
